SEQUENCE LISTING (2 sequences)

SEQ ID NO: 1 - DNA, 1743 bp, Artificial Sequence
  Nucleotide sequence of a pine longifolene synthesis gene
atggcccagatctccatcggcgcccccctctccgccgaggtcaacggcgcctgcatcaacacccaccaccacggcaacctctgggacgactacttcatccagtccctcaagtccccctacgaggcccccgagtgccacgagcgctgcgagaagatgatcgaggaggtcaagcacctcctcctctccgagatgcgcgacggcaacgacgacctcatcaagcgcctccagatggtcgacatcttcgagtgcctcggcatcgaccgccacttccaccacgagatccaggccgccctcgactacgtctaccgctactggaacgagctcgagggcatcggcgtcggcacccgcgactccctcaccaaggacctctacgccaccggcctcggcttccgcgccctccgcctccaccgctacaacgtctcctccgccgtcctcgagaacttcaagaacgagaacggcctcttcttccactcctccgccgtccaggaggaggaggtccgctgcatgctcaccctcctccgcgcctccgagatctccttccccggcgagaaggtcatggacgaggccaaggccttcgccaccgagtacctcaaccagctcctcacccgcgtcgacatcaccgaggtcggcgagaacctcctccgcgaggtccgctacgccctcgacttcccctggtactgctccgtcccccgctgggaggcccgctccttcatcgagatcttcggccagaacaactcctggctcaagtccaccatgaacaagaaggtcctcgagctcgccaagctcgacttcaacatcctccagtccgcccaccagcgcgagctccagctcctctcccgctggtggtcccagtccgacatcgagaagcagaacttctaccgcaagcgccacgtcgagttctacttctggatggtcatcggcaccttcgagcccgagttctcctcctcccgcatcgccttcgccaagatcgccaccctcatgaccatcctcgacgacctctacgacacccacggcaccctcgagcagctcaagatcttcaccgaggccgtcaagcgctgggacctctccctccaggaccgcctccccgactacatcaagatcaccctcgagttcttcttcaacacctccaacgagctcaacgccgaggtcgccaagatgcaggagcgcgacatgtccgcctacatccgcaaggccggctgggagcgctacatcgagggctacatgcaggagtccgagtggatggccgcccgccacgtccccaccttcgacgactacatgaagaacggcaagcgctcctccggcatgtgcatcctcaacctctactccctcctcctcatgggccagctcgtccccgacaacatcctcgagcagatccacctcccctccaagatccacgagctcgtcgagctcaccgcccgcctcgtcgacgactccaaggacttccaggccaagaaggacggcggcgagttcgcctccggcaccgagtgctacctcaaggagaagcccgagtgcaccgaggaggacgccatgaaccacctcatcggcctcctcaacctcaccgccatggagctcaactgggagttcgtcaagcacgacggcgtcgccctctgcctcaagaagttcgtcttcgaggtcgcccgcggcctccgcttcatctacaagtaccgcgacggcttcgactactccaacgaggagatgaagtcccagatcaccaagatcctcatcgaccaggtccccatctaa

SEQ ID NO: 2 - protein, 580 aa, Artificial Sequence
  Amino acid sequence of longifolene synthase TPS
MAQISIGAPLSAEVNGACINTHHHGNLWDDYFIQSLKSPYEAPECHERCEKMIEEVKHLLLSEMRDGNDDLIKRLQMVDIFECLGIDRHFHHEIQAALDYVYRYWNELEGIGVGTRDSLTKDLYATGLGFRALRLHRYNVSSAVLENFKNENGLFFHSSAVQEEEVRCMLTLLRASEISFPGEKVMDEAKAFATEYLNQLLTRVDITEVGENLLREVRYALDFPWYCSVPRWEARSFIEIFGQNNSWLKSTMNKKVLELAKLDFNILQSAHQRELQLLSRWWSQSDIEKQNFYRKRHVEFYFWMVIGTFEPEFSSSRIAFAKIATLMTILDDLYDTHGTLEQLKIFTEAVKRWDLSLQDRLPDYIKITLEFFFNTSNELNAEVAKMQERDMSAYIRKAGWERYIEGYMQESEWMAARHVPTFDDYMKNGKRSSGMCILNLYSLLLMGQLVPDNILEQIHLPSKIHELVELTARLVDDSKDFQAKKDGGEFASGTECYLKEKPECTEEDAMNHLIGLLNLTAMELNWEFVKHDGVALCLKKFVFEVARGLRFIYKYRDGFDYSNEEMKSQITKILIDQVPI

(19) United States
(12) Patent Application Publication
(10) Pub. No.: US 2024/0251802 A1
(43) Pub. Date: Aug. 1, 2024

(54) USE OF SATIVENE, AND RECOMBINANT BIOCONTROL FUNGUS AND MUSCARDINE CADAVER AND USE THEREOF

(71) Applicant: ZHEJIANG UNIVERSITY, Zhejiang (CN)

(72) Inventors: Weiguo FANG, Zhejiang (CN); Dan Tang, Zhejiang (CN)

(21) Appl. No.: 18/014,692

(22) PCT Filed: Jan. 27, 2022
(86) PCT No.: PCT/CN2022/074175

§ 371 Date: Jan. 5, 2024

(30) Foreign Application Priority Data
Jan. 21, 2022 (CN) 202210069879.X

(51) Int. Cl. A01N 63/30; A01N 45/02; A01N 63/14; A01P 7/04; A01P 19/00; C12N 15/80; C12P 15/00

(52) U.S. Cl. CPC A01N 63/30; A01N 45/02; A01N 63/14; A01P 7/04; A01P 19/00; C12N 15/80; C12P 15/00

(57) ABSTRACT

The present disclosure provides use of sativene, and a recombinant biocontrol fungus and a muscardine cadaver and use thereof, belonging to the technical field of genetic engineering. The present disclosure provides use of sativene in insect attraction and/or pest control. The sativene are attractive to insects. A higher concentration of the sativene has a stronger attraction on mosquito and Drosophila larvae; the Drosophila larvae respond to the sativene even at a level as low as 10⁻¹¹ g. The sativene also be attractive to Galleria mellonella larvae. The present disclosure further provides a recombinant biocontrol fungus, including a recombinant fungal expression plasmid, where the recombinant fungal expression plasmid is inserted with a pine longifolene synthesis gene. In the present disclosure, the recombinant biocontrol fungus has increased synthetic amounts and volatilized amounts of the longifolene and sativene, resulting in an enhanced attraction to insects.

### CROSS REFERENCE TO RELATED APPLICATION

This patent application claims the benefit and priority of Chinese Patent Application No. 202210069879.X, filed with the China National Intellectual Property Administration on Jan. 21, 2022, the disclosure of which is incorporated by reference herein in its entirety as part of the present application.

### REFERENCE TO SEQUENCE LISTING

A computer readable file titled “GWPCTP20220902136_Sequence_Listing_ST25.txt”, created on 5 Jan. 2023, with a file size of about 7,600 bytes, contains the sequence listing for this application, has been filed with this application, and is incorporated by reference herein in its entirety.

### TECHNICAL FIELD

The present disclosure belongs to the technical field of genetic engineering, and particularly relates to use of sativene, and a recombinant biocontrol fungus and a muscardine cadaver and use thereof.

### BACKGROUND

Fungi are the most common insect pathogens, and more than 1,000 fungi have been found can infect insects. Among them, fungi of the genus Metarhizium, Beauveria, Paecilomyces, and Verticillium in the class Hyphomycetes have been developed into a variety of fungal inoculants for controlling agricultural, forestal, and sanitary pests and diseases transmitted by these pests (Zhao H. Lovett B, and Fang W. Genetically Engineering Entomopathogenic Fungi. Adv Genet. 2016, 94: 137-63). At present, there are more than 60 kinds of filamentous fungal insecticides registered around the world, serving as a source of new technologies and products for biological pest control. The existing research has found that fungal insecticides based on the class Hyphomycetes kill pests primarily through the contagious infection of body wall. This process involves the adsorption, germination and penetration of spores on the insect body wall. Then fungi reproduce and secrete toxins in the host's hemocoel, causing death to the host; and the fungi grow from the resulting muscardine cadaver and produces a large number of the spores on the body surface of the muscardine cadaver. The spores may spread in the environment and infect other healthy pests for further or even long-term pest control. There are four main factors that determine a field pest control efficiency of the fungal insecticides: an inoculation rate (a proportion of pests with spores attached to their body wall) when a fungal insecticide is applied, a tolerance to environmental stress before the spores invade the pests, an insecticidal rate, and a spread rate of newly-formed spores on the muscardine cadaver. On a basis of in-depth researches of the pathogenicity and stress resistance mechanism of entomopathogenic fungi, genetic engineering was used by predecessors to improve the insecticidal rate or stress resistance of the entomopathogenic fungi by, and to provide a number of genetically improved strains for enhancing an efficiency of the fungal insecticides (Zhao H, Lovett B, and Fang W. Genetically Engineering Entomopathogenic Fungi. Adv Genet. 2016, 94: 137-63).

According to the characteristics of life activities of pests, people use fungal insecticides in different ways. For pests with weak activity, such as lepidopteran larvae, the existing methods are to produce a large number of spores through fermentation and the like, and fungal insecticide preparations such as oil/powder and granules are prepared to be mainly released in the field by spraying and soil application. During the application, fungal spores may come into direct contact with the pests, or may spray in the environment (leaves or soil) and inoculated to the pests, due to activities of them. Other methods have also been tried to increase the inoculation rate. For example, fungal insecticides are loaded on insect enemies (such as predatory mites), then the natural enemies transmit spores to target pests, thus achieving the joint control of pests by the fungal insecticides and natural enemies (Shengyong Wu, Qingpo Yang, Changchun Xu, Xuenong Xu, and Zhongren Lei. Rescarch Prospect in Interactions between Entomopathogenic Fungi and Predatory Mites and Their Combined Applications. Chinese Journal of Biological Control. 2019, 35: 127-133). For pests move by means of flying that has strong mobility, such as mosquitoes, the spraying has a low inoculation efficiency. According to the activity characteristics of mosquitoes, some methods have been designed for application of fungal insecticides. For example, the fungal insecticides are pre-sprayed on a surface of a solid medium (such as black cotton cloth, mosquito nets, and walls), and the solid medium is housed indoors and in mosquito-entry passages (such as caves of a traditional African house) (Lovett B, Bilgo E, Millogo S A, Ouattarra A K, Sare I, Gnambani E J, Dabire R K, Diabate A. St Leger R J. Transgenic Metarhizium rapidly kills mosquitoes in a malaria-endemic region of Burkina Faso. Science. 2019 May 31; 364 (6443): 894-897). In addition, there have been attempts to increase the mosquito inoculation rate by placing devices containing mosquito attractants and fungal insecticides outdoors.

Overall, inoculation efficiency determines the effectiveness of fungal insecticides against flying pests such as mosquitoes. The various strategies adopted by the existing application methods have improved the inoculation rate to a certain extent. However, these application methods still cannot meet the requirements of pest control, with a need of the inoculation efficiency to be further improved.

### SUMMARY

In view of this, an objective of the present disclosure is to provide use of sativene, and a recombinant biocontrol fungus and a muscardine cadaver and use thereof. The sativene, the recombinant biocontrol fungus, and the muscardine cadaver each can attract insects and improve the insect inoculation rate.

The present disclosure provides use of sativene in insect attraction and/or pest control, where the sativene has a structure shown in Formula I;

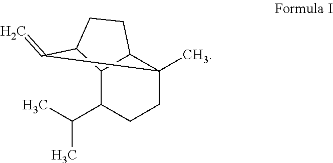

Preferably, the insect includes one or more of Drosophila, Galleria mellonella, and mosquitoes.

The present disclosure further provides a recombinant biocontrol fungus, including a recombinant fungal expression plasmid, where a synthesis gene of pine longifolene is inserted into the recombinant fungal expression plasmid.

Preferably, the synthesis gene of pine longifolene has a nucleotide sequence set forth in SEQ ID NO: 1.

Preferably, an original fungus of the recombinant biocontrol fungus includes Metarhizium.

The present disclosure further provides a muscardine cadaver, which is infected with the recombinant biocontrol fungus.

The present disclosure further provides use of the recombinant biocontrol fungus or the muscardine cadaver in insect attraction and/or pest control.

Preferably, the insect includes one or more of Drosophila, Galleria mellonella, and mosquitoes.

The present disclosure further provides use of the recombinant biocontrol fungus or the muscardine cadaver in preparation of sativene, where the sativene has a structure shown in formula I;

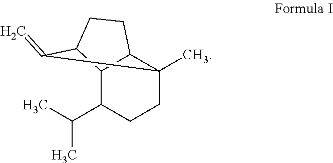

The present disclosure further provides an insect attractant, where an active ingredient of the insect attractant includes sativene or the recombinant biocontrol fungus or the muscardine cadaver; and the sativene has a structure shown in formula I;

The present disclosure provides use of sativene in insect attraction and/or pest control. The sativene are attractive to insects and can improve the inoculation rate of insects. The present studies have found that, a higher concentration of the sativene has a stronger attracting effect on mosquito and Drosophila larvae. And the Drosophila larvae respond to the sativene even at a level as low as 10⁻¹¹ g. The sativene also has insect attracting properties to Galleria mellonella larvae.

The present disclosure further provides a recombinant biocontrol fungus, including a recombinant fungal expression plasmid, where a synthesis gene of pine longifolene has been inserted into the recombinant fungal expression plasmid. In the present disclosure, the recombinant biocontrol fungus has high synthetic amounts of the longifolene and sativene and the amounts of the longifolene and sativene volatilized by spores of the fungus is large, resulting in an enhanced attraction to insects, so as to increase the inoculation rate of insects. The present disclosure provides a new solution for solving the low inoculation efficiency of the fungal insecticides when controlling pests such as mosquitoes, and shows broad application prospects.

### BRIEF DESCRIPTION OF THE DRAWINGS

FIGS. 1A, 1B, 1C, and 1D show a device to test the attraction of healthy insects by a muscardine cadaver obtained by infecting old larvae of Galleria mellonella with Metarhizium robertsii in Example 1, and a test result thereof; specifically, FIG. 1A shows a schematic diagram of a device for detecting the attraction of a Galleria mellonella muscardine cadaver to insects by a Two-way choice method; where gray and white insects represent the muscardine cadaver and freeze-to-death insects (control), respectively. FIG. 1B shows a schematic diagram of a device for detecting the attraction of Galleria mellonella muscardine cadaver to Drosophila larvae by the Two-way choice method; where gray and white insects represent the muscardine cadaver and death insects (control), respectively, the middle part is a release place of healthy Drosophila larvae, and healthy insects entering the left and right sides represent an impact of the muscardine cadaver and the freeze-to-death insects (control), respectively. FIG. 1C shows a response index of Galleria mellonella larvae, Drosophila larvae, and Aedes albopictus adults to the muscardine cadaver obtained by infecting the Galleria mellonella with Metarhizium robertsii. FIG. 1D shows a schematic diagram of a device for detecting the attraction of Galleria mellonella muscardine cadaver to Aedes albopictus adults by the Two-way choice method; where gray and white insects represent the muscardine cadaver and freeze-to-death insects (control), respectively, and healthy Aedes albopictus are placed in a tube 1 in the middle part;

FIGS. 2A, 2B, 2C, and 2D show an influence of different concentrations of longifolene and sativene on insect behavior; where FIG. 2A shows an influence of different concentrations of longifolene on the behavior of Drosophila larvae; FIG. 2B shows an influence of different concentrations of sativene on the behavior of Drosophila larvae; FIG. 2C shows an influence of different concentrations of longifolene on the behavior of Aedes albopictus adults; and FIG. 2D shows an influence of different concentrations of sativene on the behavior of Aedes albopictus adults;

FIG. 3 shows an influence of longifolene and sativene on the behavior of Galleria mellonella larvae;

FIGS. 4A and 4B show construction of a transgenic strain Mr-Tps; where FIG. 4A shows a profile of a pPK2-bar-gpd-GFP-Tps vector; and FIG. 4B shows transcription expression of a Tps gene, where Act is a reference gene;

FIGS. 5A, 5B, 5C, and 5D show volatilized amounts of longifolene and sativene and their effects on insect behavior in muscardine cadavers of wild-type (WT) and transgenic strain Mr-Tps under different culture conditions; where FIG. 5A shows the WT and transgenic strain Mr-Tps cultured on the muscardine cadaver; FIG. 5B shows the WT and transgenic strain Mr-Tps cultured on a potato dextrose agar (PDA) medium; FIG. 5C shows the WT and transgenic strain Mr-Tps cultured on a fermentation medium; FIG. 5D shows an influence of the muscardine cadavers formed by Mr-Tps and WT infections on the insect behavior; and ** represents an extremely significant difference (n=6, P<0.01, Wilcoxon signed-rank test);

FIGS. 6A and 6B show a preference analysis of Aedes albopictus and Drosophila larvae for WT and Mr-Tps under different culture conditions; where FIG. 6A shows a preference of the WT and Mr-Tps for Aedes albopictus grown on the PDA, cadavers, and fermentation medium; ** represents an extremely significant difference (n=6, P<0.01, Wilcoxon signed-rank test); and FIG. 6B shows a preference of the WT and Mr-Tps for Drosophila larvae grown on the PDA, cadavers, and fermentation medium; and ** represents an extremely significant difference (n=6, P<0.01, Wilcoxon signed-rank test); and

FIGS. 7A and 7B show an inoculation rate and an inoculation amount of the WT and Mr-Tps onto Aedes albopictus; where FIG. 7A shows an inoculation rate of Aedes albopictus by the WT and Mr-Tps with an inoculation amount of 1×10⁸ spores in total, herein the inoculation rate=the number of inoculated mosquitoes/the total number of mosquitoes; and ** represents an extremely significant difference (n=6, P<0.01, Wilcoxon signed-rank test); and FIG. 7B shows an inoculation rate of Aedes albopictus by the WT and Mr-Tps with 1×10⁸ spores in total, herein the inoculation amount is the number of spores inoculated onto Aedes albopictus; and * represents a significant difference (n=6, P<0.05, Wilcoxon signed-rank test).

### DETAILED DESCRIPTION OF THE EMBODIMENTS

The present disclosure provides use of sativene in insect attracting and/or pest control, where the sativene has a chemical structure shown in Formula I;

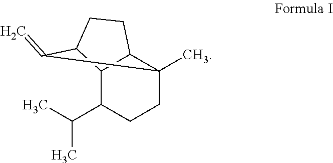

In the present disclosure, there is no special limitation on a source of the sativene, and the sativene can be prepared by conventional methods in the art or derived from commercially available sources. The sativene has insect attraction; the insect includes preferably one or more of Drosophila, mosquitoes, and Galleria mellonella.

The present disclosure further provides a recombinant biocontrol fungus, including a recombinant fungal expression plasmid, where the recombinant fungal expression plasmid is inserted with a pine longifolene synthesis gene.

In the present disclosure, the recombinant biocontrol fungus has stronger insect attraction and insecticidal efficacy than those of original biocontrol fungi.

In the present disclosure, the pine longifolene synthesis gene has a nucleotide sequence set forth in SEQ ID NO: 1, specifically as follows:

atggcccagatctccatcggcgcccccctctccgccgaggtcaacggcgcctgcatcaacacccaccaccacggcaacctct
gggacgactacttcatccagtccctcaagtccccctacgaggcccccgagtgccacgagcgctgcgagaagatgatcgaggaggtcaag
cacctcctcctctccgagatgcgcgacggcaacgacgacctcatcaagcgcctccagatggtcgacatcttcgagtgcctcggcatcgacc
gccacttccaccacgagatccaggccgccctcgactacgtctaccgctactggaacgagctcgagggcatcggcgtcggcacccgcgac
tccctcaccaaggacctctacgccaccggcctcggcttccgcgccctccgcctccaccgctacaacgtctcctccgccgtcctcgagaactt
caagaacgagaacggcctcttcttccactcctccgccgtccaggaggaggaggtccgctgcatgctcaccctcctccgcgcctccgagatc
tccttccccggcgagaaggtcatggacgaggccaaggccttcgccaccgagtacctcaaccagctcctcacccgcgtcgacatcaccga
ggtcggcgagaacctcctccgcgaggtccgctacgccctcgacttcccctggtactgctccgtcccccgctgggaggcccgctccttcatc
gagatcttcggccagaacaactcctggctcaagtccaccatgaacaagaaggtcctcgagctcgccaagctcgacttcaacatcctccagt
ccgcccaccagcgcgagctccagctcctctcccgctggtggtcccagtccgacatcgagaagcagaacttctaccgcaagcgccacgtc
gagttctacttctggatggtcatcggcaccttcgagcccgagttctcctcctcccgcatcgccttcgccaagatcgccaccctcatgaccatcc
tcgacgacctctacgacacccacggcaccctcgagcagctcaagatcttcaccgaggccgtcaagcgctgggacctctccctccaggacc
gcctccccgactacatcaagatcaccctcgagttcttcttcaacacctccaacgagctcaacgccgaggtcgccaagatgcaggagcgcga
catgtccgcctacatccgcaaggccggctgggagcgctacatcgagggctacatgcaggagtccgagtggatggccgcccgccacgtcc
ccaccttcgacgactacatgaagaacggcaagcgctcctccggcatgtgcatcctcaacctctactccctcctcctcatgggccagctcgtc
cccgacaacatcctcgagcagatccacctcccctccaagatccacgagctcgtcgagctcaccgcccgcctcgtcgacgactccaaggac
ttccaggccaagaaggacggcggcgagttcgcctccggcaccgagtgctacctcaaggagaagcccgagtgcaccgaggaggacgcc
atgaaccacctcatcggcctcctcaacctcaccgccatggagctcaactgggagttcgtcaagcacgacggcgtcgccctctgcctcaaga
agttcgtcttcgaggtcgcccgcggcctccgcttcatctacaagtaccgcgacggcttcgactactccaacgaggagatgaagtcccagatc
accaagatcctcatcgaccaggtccccatctaa.

In the present disclosure, a protein encoded by the pine longifolene synthesis gene is longifolene synthase TPS, with an amino acid sequence set forth in SEQ ID NO: 2, specifically as follows:

MAQISIGAPLSAEVNGACINTHHHGNLWDDYFIQSLKSPYEAPECHERCEKMIEE
VKHLLLSEMRDGNDDLIKRLQMVDIFECLGIDRHFHHEIQAALDYVYRYWNELEGIGVG
TRDSLTKDLYATGLGFRALRLHRYNVSSAVLENFKNENGLFFHSSAVQEEEVRCMLTLLR
ASEISFPGEKVMDEAKAFATEYLNQLLTRVDITEVGENLLREVRYALDFPWYCSVPRWEA
RSFIEIFGQNNSWLKSTMNKKVLELAKLDFNILQSAHQRELQLLSRWWSQSDIEKQNFY
RKRHVEFYFWMVIGTFEPEFSSSRIAFAKIATLMTILDDLYDTHGTLEQLKIFTEAVKRWD
LSLQDRLPDYIKITLEFFFNTSNELNAEVAKMQERDMSAYIRKAGWERYIEGYMQESEW
MAARHVPTEDDYMKNGKRSSGMCILNLYSLLLMGQLVPDNILEQIHLPSKIHELVELTAR
LVDDSKDFQAKKDGGEFASGTECYLKEKPECTEEDAMNHLIGLLNLTAMELNWEFVKH
DGVALCLKKFVFEVARGLRFIYKYRDGFDYSNEEMKSQITKILIDQVPI.

In the present disclosure, a protein sequence of the longifolene synthase TPS of Pinus sylvestris (Genbank accession number: ABV44454) is queried on NCBI (www.ncbi.nlm.nih.gov/). According to the service provided by Codon Usage Database (www.kazusa.or.jp/codon/), a codon preference of Metarhizium robertsii is obtained, and a codon type with the maximum frequency is selected to obtain a coding sequence of the TPS protein.

In the present disclosure, an original fungus of the recombinant biocontrol fungus preferably includes Metarhizium, more preferably Metarhizium robertsii.

In the present disclosure, an original plasmid of the recombinant fungal expression plasmid is preferably pPK2-bar-gpd-GFP; and insertion sites of the pine longifolene synthesis gene in the recombinant fungal expression plasmid are preferably BamH I and EcoR V. There is no special limitation on methods for constructing the recombinant fungal expression plasmid, and conventional methods in the art can be used.

In the present disclosure, there is no special limitation on methods for constructing the recombinant biocontrol fungus, and conventional methods in the art can be used.

The present disclosure further provides a muscardine cadaver, infected with the recombinant biocontrol fungus.

In the present disclosure, the muscardine cadaver is preferably prepared by the following method:
infecting recipient larvae using the recombinant biocontrol fungus, and after the recipient larvae are infected to die, the resulting cadavers are disinfected and subjected to moisturized culture to form muscardine cadavers covered with spores.

In the present disclosure, the recipient larvae are preferably last-instar larvae of the Galleria mellonella. A process of infecting recipient larvae using the recombinant biocontrol fungus includes infecting the recipient larvae using a spore suspension of the recombinant biocontrol fungus; and the spore suspension of the recombinant biocontrol fungus has preferably 1×10⁷ spores/ml of the recombinant biocontrol fungus by concentration. A disinfectant used in the disinfection is preferably a sodium hypochlorite solution having preferably 0.05% of sodium hypochlorite by mass concentration.

The present disclosure further provides use of the recombinant biocontrol fungus or the muscardine cadaver in insect attraction and/or pest control.

In an example of the present disclosure, the insect may include one or more of Drosophila, Galleria mellonella, and mosquitoes.

The present disclosure further provides use of the recombinant biocontrol fungus or the muscardine cadaver in preparing sativene, where the sativene has a structure shown in formula I;

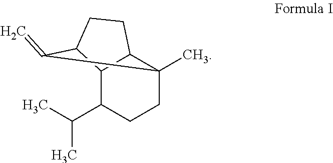

The present disclosure further provides an insect attractant, where an active ingredient of the insect attractant includes sativene or the recombinant biocontrol fungus or the muscardine cadaver as described above; and the sativene has a structure shown in formula I;

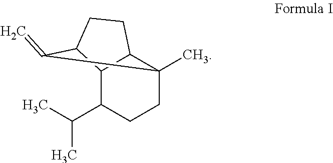

In the present disclosure, sativene has an effective dosage of preferably 10⁻¹¹ g to 10⁻⁵ g for Drosophila larvae in a space of 64 cm³, and 105 g has the strongest attractive effect on the Drosophila larvae; and the sativene has an effective dosage of 10⁻⁹ g to 10⁻⁵ g for Aedes albopictus adults in a space of 640 cm³, and 10⁻⁵ g has the strongest attracting effect on the mosquitoes.

The present disclosure further provides an insecticide or an insecticidal device, including the insect attractant.

The technical solutions of the present disclosure will be described below clearly and completely in conjunction with the examples of the present disclosure.

### Example 1 Attraction of Muscardine Cadavers Derived from Metarhizium robertsii-Infected Older Larvae of Galleria mellonella to Healthy Insects

### 1. Detection Methods

(1) Preparation of Muscardine Cadavers Derived from Metarhizium robertsii-Infected Older Larvae of Galleria mellonella

Metarhizium robertsii was cultured on PDA for 14 d, prepared into a spore suspension (1×10⁷ spores/ml) with Triton-X-100 solution (0.01%), and then used to infect older larvae of Galleria mellonella. After the larvae were infected and died, the resulting cadavers were disinfected with sodium hypochlorite solution (0.05%), and subjected to moisturized culture to form muscardine cadavers covered with spores.

(2) Two-Way Choice Method for Detecting Attraction of Galleria mellonella Muscardine Cadavers to Drosophila Healthy Larvae

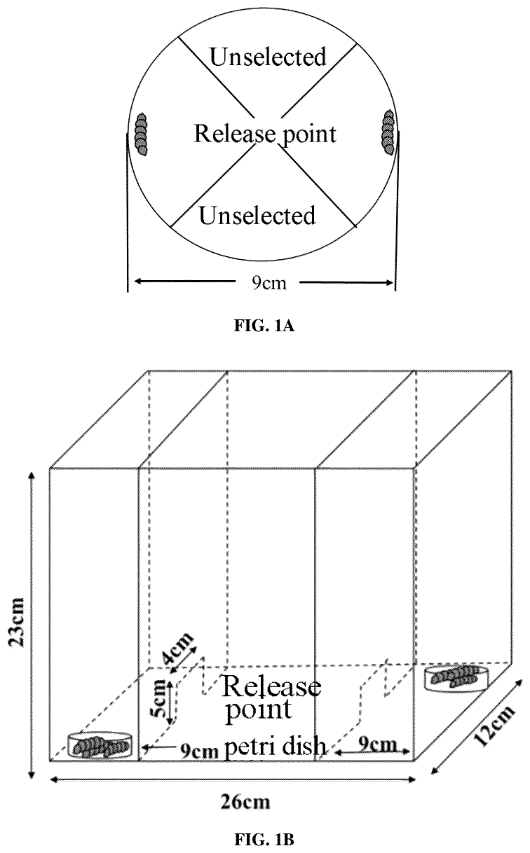

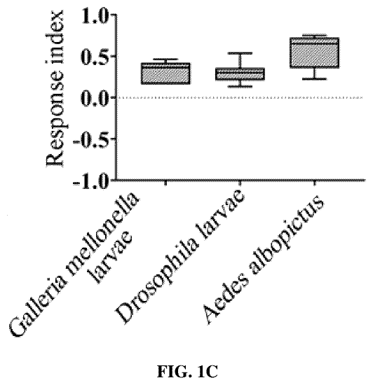

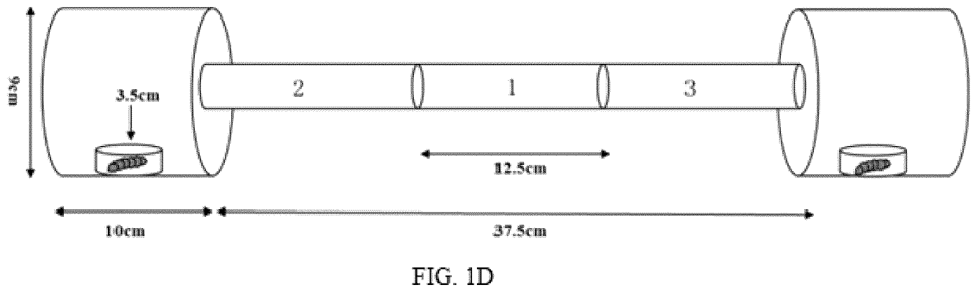

Two-way choice method was used to detect the attraction of Galleria mellonella muscardine cadavers to Drosophila healthy larvae, with the device shown in FIG. 1A. New freezing-killed Galleria mellonella larvae were kept at a room temperature for 20 min(control) and one of the resulting Galleria mellonella muscardine cadaver was placed on both sides of a 9 cm petri dish (containing 2% water agar), and then 20 healthy 3-instar Canton-S Drosophila larvae were placed in the middle part of the petri dish. After 10 min, photographing was conducted to record the selection of Drosophila larvae, and a Response index was calculated [the Response index=(the number of Drosophila larvae selecting Galleria mellonella muscardine cadaver−the number of Drosophila larvae selecting control Galleria mellonella larvae)/the total number of Drosophila larvae (20)]. The experiment was repeated 6 times.

(3) Detection on Influence of Galleria mellonella Muscardine Cadavers to Galleria mellonella Healthy Larvae

An improved Two-way choice method was used to detect the attraction of Galleria mellonella muscardine cadavers to Galleria mellonella healthy larvae, with the device shown in FIG. 1B. Three freeze-killed Galleria mellonella larvae (control) and three the resulting Galleria mellonella muscardine cadavers were placed in 9 cm petri dishes, and the petri dishes were placed diagonally in the device; 40 healthy Galleria mellonella larvae were released at the center point of the device, and placed at room temperature in dark. The selection of healthy Galleria mellonella larvae was recorded after 1 h. A Response index was calculated [the Response index=the number of Galleria mellonella larvae selecting Galleria mellonella muscardine cadavers−the number of Galleria mellonella larvae selecting freeze-killed Galleria mellonella larvae)/the total number of Galleria mellonella larvae]. The experiment was repeated 6 times.

(4) Detection on Attraction of Galleria mellonella Muscardine Cadavers to Adult Mosquitoes

The detection on attraction of muscardine cadavers to mosquito (Aedes albopictus) adults was conducted according to a relevant literature (Robinson Ailic, Busula Annette O, Voets Mirjam A, Beshir Khalid B, Caulfield John C. Powers Stephen J, Verhulst Niels O, Winskill Peter, Muwanguzi Julian, Birkett Michael A, Smallegange Renate C, Masiga Daniel K, Mukabana W Richard, Sauerwein Robert W, Sutherland Colin J, Bousema Teun, Pickett John A, Takken Willem, Logan James G, de Boer Jetske G. Plasmodium-associated changes in human odor attract mosquitoes. Proc Natl Acad Sci USA 2018; 115: 4215), and the device used was shown in FIG. 1D. One of freeze-killed Galleria mellonella larvae (control) and one of the resulting Galleria mellonella muscardine cadaver were placed in a 3.5 cm ampoule, and placed in cylinders on both sides of the device. 10 female mosquitoes without blood meal 3 d to 5 d after eclosion were placed in the middle of a tube 1. Openings at both ends of the tube 1 were covered with gauze, and the tube was placed in a refrigerator at 4° C. for 3 min to reduce mosquito activities. Then the tube 1 was connected to tubes 2 and 3. The whole device was placed in a dark incubator at 26° C., and the selection of mosquitoes was recorded after 10 h. A Response index was calculated [the Response index=the number of mosquitoes selecting Galleria mellonella muscardine cadavers−the number of mosquitoes selecting freeze-killed Galleria mellonella larvae)/the total number of mosquitoes].

### 2. Results

A behavioral assay based on the Two-way choice method showed that, the muscardine cadavers derived from Metarhizium robertsii-infected Galleria mellonella larvae had an attractive effect on each of the Drosophila larvae, Galleria mellonella larvae, and Aedes albopictus adults (FIG. 1C).

### Example 2 Volatile Compounds Attractive to Pests Produced by Muscardine Cadavers

### 1. Analysis Methods for Muscardine Cadaver-Derived Volatile Compounds

(1) Extraction method: five muscardine cadavers derived from Metarhizium robertsii-infected Galleria mellonella larvae were placed in a 20 ml injection vial, and a 50/30 μm DVB/CAR/PDMS extraction tip was inserted into the injection vial to conduct absorption-extraction for 50 min by heating in a 45° ° C. water bath.

(2) SPME-GC-MS analysis method: after the extraction, a sample was manually injected, and an injection port was desorptede at 250° C. for 3 min. A chromatographic column was a 30 m×0.25 mm×0.25 μm DB-5MS chromatographic column; a column temperature was started at 35° C. and held for 5 min, then increased to 145° C. at 2° C./min, and then increased to 250° C. at 15° C./min (holding for 10 min). According to a total ion current chromatogram, analysis was conducted combined with the mass spectrum characteristic data of each chromatographic peak, and each volatile component was identified by comparison with a mass spectrometry library (NIST05) to preliminarily identify the substance type. A standard was purchased according to the CAS number of a substance provided by the mass spectrometry library; the retention time and mass spectrometry characteristics of the standard were further analyzed by GC-MS. And the retention time and mass spectrum characteristics of a to-be-identified compound was compared with those of the standard. If these characteristics were consistent, it was confirmed that the to-be-identified compound and the standard were the same substance.

### 2. Results

13 volatile substances were produced by the muscardine cadavers derived from Metarhizium robertsii-infected Galleria mellonella larvae (Table 1). By comparing with the standards, it was found that among the 5 compounds with the highest proportion, 4 were known compounds (longifolene, sativene, β-farnesene, and geosmin), where a compound with the highest proportion was an unknown sesquiterpene. An ability of the β-farnesene and geosmin to attract insects and a mechanism by which insects sense these two compounds had been elucidated. In the present disclosure, the insect attraction of the longifolene and sativene was studied in depth.

TABLE 1
Volatile substances produced by Metarhizium robertsii-infected
Galleria mellonella and their respective proportions

| Compound | Peak area percentage (%) |
|---|---|
| Fail to identify | 65.21 |
| Longifolene | 8.61 |
| Geosmin | 7.15 |
| Sativene | 4.01 |
| Trans-β-farnesene | 3.37 |
| 2,5-diethylpyrazine | 2.70 |
| (−)-limonene | 2.17 |
| 2,3,5,8-tetramethyldecane | 1.85 |
| 6-methyloctadecane | 1.62 |
| 2,6,10-trimethyltetradecane | 1.34 |
| α-selinene | 0.98 |
| caryophyllin | 0.49 |
| n-tetradecane | 0.48 |

### Example 3 Insect Attraction of Sativene and Longifolene

### 1) Analysis Method

Longifolene and sativene standards were purchased from Sigma-Aldrich with a purity of 99%.

The two-way choice method for detecting the attraction of longifolene and sativene to Drosophila larvae had a process similar to detecting the attraction of muscardine cadaver to Drosophila larvae. The muscardine cadaver was replaced by circular filter paper with a 5 mm diameter (containing 10 μl of different concentrations solutions of longifolene or sativene), and a control was a filter paper containing 10 μl of n-hexane solvent. Similarly, the method for detecting the attraction of muscardine cadavers to Galleria mellonella larvae and mosquitoes adults was also used to analyze the attraction of longifolene and sativene to these two kinds of insects. In the Galleria mellonella attractive device, the muscardine cadaver was replaced by cotton balls with about 2 cm diameter (containing 100 μl different concentrations solutions of longifolene or sativene), and a control was cotton balls containing 100 μl of n-hexane solvent. In the mosquito attractive device, the muscardine cadaver was replaced by cotton balls with about 1 cm diameter (containing 100 μl of different concentrations solutions of longifolene or sativene), and a control was cotton balls containing 100 μl of n-hexane solvent.

### 2) Results

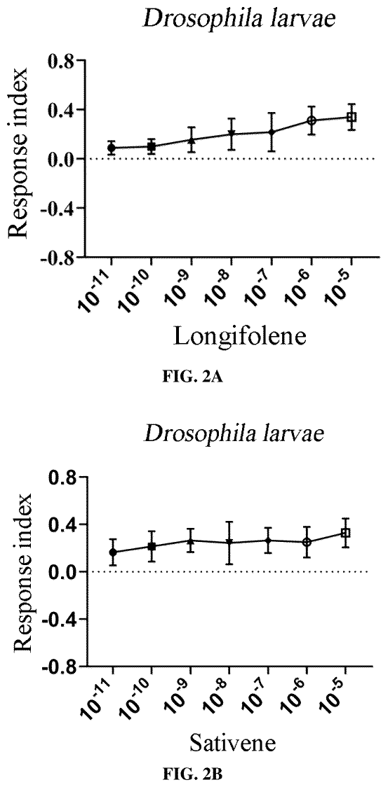

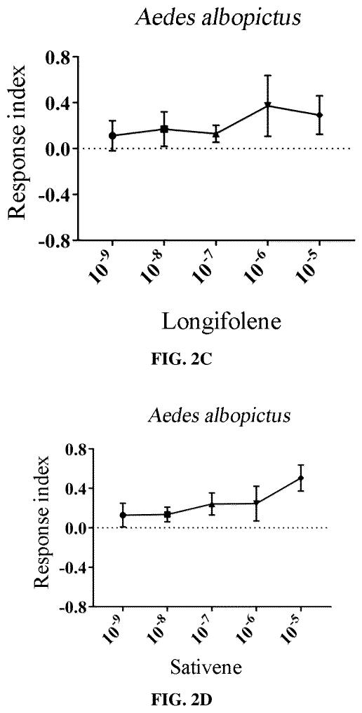

To further explore a concentration range of Drosophila larvae responding to longifolene and sativene, seven content gradients of longifolene and sativene were set: 10⁻⁵ g, 10⁻⁶ g, 10⁻⁷ g, 10⁻⁸ g, 10⁻⁹ g, 10⁻¹⁰ g, and 10⁻¹¹ g to conduct behavioral experiments. The results were shown in FIG. 2A and FIG. 2B, and the results showed that a higher concentration of the longifolene and sativene had a stronger attraction on Drosophila larvae; the Drosophila larvae responded to the longifolene and sativene even at a level as low as 10⁻¹¹ g.

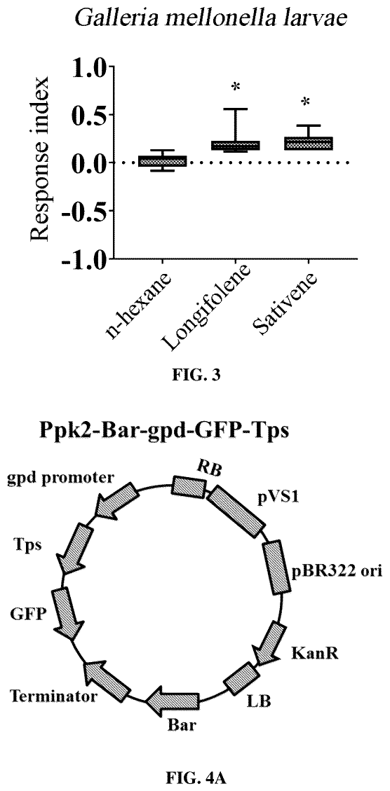

Similarly, five content gradients of longifolene and sativene, i.e. 10⁻⁵ g, 10⁻⁶ g, 10⁻⁷ g, 10⁻⁸ g, and 10⁻⁹ g were set up to explore a concentration range of mosquitoes respond to the longifolene and sativene. The results were shown in FIG. 2C and FIG. 2D, indicating that 10⁻⁶ g longifolene had the strongest attraction on mosquitoes, and mosquitoes had little response to 10⁻⁹ g longifolene. For sativene, a higher concentration of the sativene had a stronger attraction on mosquitoes. In addition, longifolene and sativene could also attract Galleria mellonella larvae, and the results were shown in FIG. 3.

### Example 4 Construction of Recombinant Metarhizium robertsii Strain with Increased Volatilized Amounts of Sativene and Longifolene

### (1) Methods:

### 1) Gene Discovery and Synthesis

A protein sequence of the longifolene synthase Tps of Pinus sylvestris (Genbank accession number: ABV44454) was queried on NCBI (www.ncbi.nlm.nih.gov/), with a sequence information set forth in SEQ ID NO: 2. According to the service provided by Codon Usage Database (www.kazusa.or.jp/codon/), a codon preference of Metarhizium robertsii was obtained, and a codon type with the maximum frequency was selected to obtain a coding sequence of the TPS protein. The sequence was set forth in SEQ ID NO: 1, synthesized by Hangzhou Youkang Biotechnology Co., Ltd., and then ligated to a puc57-simple-TOPO vector to obtain a plasmid containing a longifolene synthase coding sequence, named puc57-simple-TOPO-PsTPS.

### 2) Construction of TPS Expression Vector and Construction of Recombinant Strain

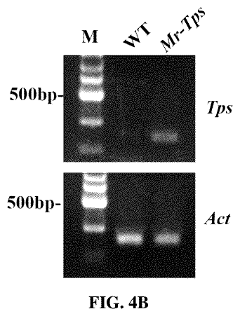

With the puc57-simple-TOPO-PsTps as a template, the Tps coding sequence was cloned by PCR using a high-fidelity DNA polymerase KOD Plus Neo (TOYOBO) with primers PsTps-CDS-FP-BamHI (ATGCCC) and PsTps-CDS-RP-EcoRV (ACTGGGG). A PCR product was digested with restriction endonucleases BamH I and EcoR V (Thermo Scientific), and ligated with a vector pPK2-bar-gpd-GFP digested with the same enzymes to obtain a Tps expression vector pPK2-bar-gpd-GFP-TPS, where vector information was shown in FIG. 4A.

The plasmid pPK2-bar-gpd-GFP-PsTps was transformed into an Agrobacterium tumefaciens strain AGL1, and then transformed into a Metarhizium robertsii strain ARSEF 2575. Transformants were initially screened by herbicide resistance selection and green fluorescent protein (GFP) observation. And it was further confirmed by PCR (with primers PsTps-CDS-FP-BamHI and PsTps-CDS-RP-EcoRV) that a Tps expression cassette was successfully integrated into the genome of the Metarhizium robertsii. RT-PCR proved that the Tps encoding gene was transcribed and expressed (FIG. 4B), and a Metarhizium robertsii strain Mr-Tps heterologously expressing the Tps gene was obtained.

### 2) Results

### (1) Transcriptional Expression of Tps Encoding Gene Proved by RT-PCR

Gel electrophoresis was conducted to detect the transcription and expression of Tps-encoding gene, and the results were shown in FIG. 4B.

### (2) More Longifolene and Sativene was Volatilized by Transgenic Strain Mr-Tps

Volatile substances produced by muscardine cadavers derived from transgenic strain Mr-Tps-infected Galleria mellonella larvae were analyzed according to the method described above. It was found (Table 2) that the muscardine cadavers produced 11 volatile substances, among which a compound with the highest proportion was longifolene, followed by farnesene, longicyclene, and sativene. Compared with muscardine cadavers derived from WT strain infection, the muscardine cadavers derived from transgenic strain Mr-Tps-infected Galleria mellonella larvae volatilized a greater variety of sesquiterpenes, including longicyclene, longipinene, and cedrene.

TABLE 2
Analyses results of volatile substances produced by
WT strain- and transgenic strain Mr-Tps-derived muscardine
cadavers under different culture conditions

| | Peak area percentage (%) | | |
|---|---|---|---|
| | Muscardine | PDA | Fermentation |
| Compound | cadaver | medium | medium |
| Longifolene | 70.70 | 67.95 | 64.53 |
| 2,5-diethylpyrazine | 12.73 | | |
| Longicyclene | 3.31 | 3.83 | 5.81 |
| Sativene | 3.06 | 2.38 | 2.29 |
| Longipinene | 2.65 | 5.56 | 6.92 |
| Fail to identify | 1.85 | | |
| 2,6-di-tert-butyl-p-cresol | 1.59 | | |
| Trans-β-farnesene | 1.25 | | |
| 2,6,11,15-tetramethylhexadecane | 1.05 | | |
| 2,7,10-trimethyl-dodecane | 1.03 | | |
| Cedrene | 0.77 | | |
| Styrene | | 17.62 | |
| n-tetradecane | | 2.66 | |
| 1,3-octadiene | | | 19.59 |

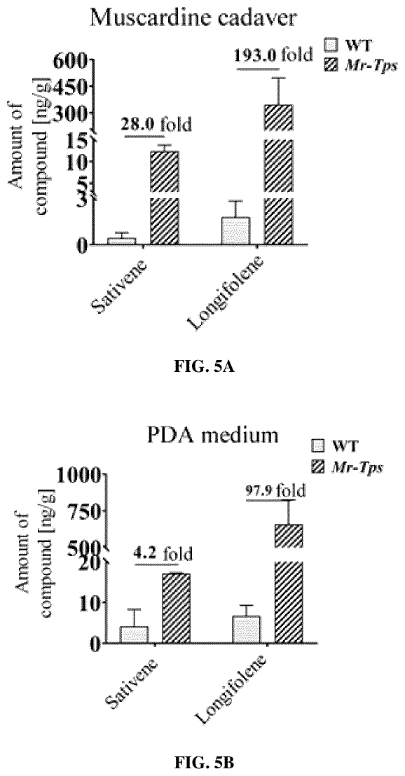

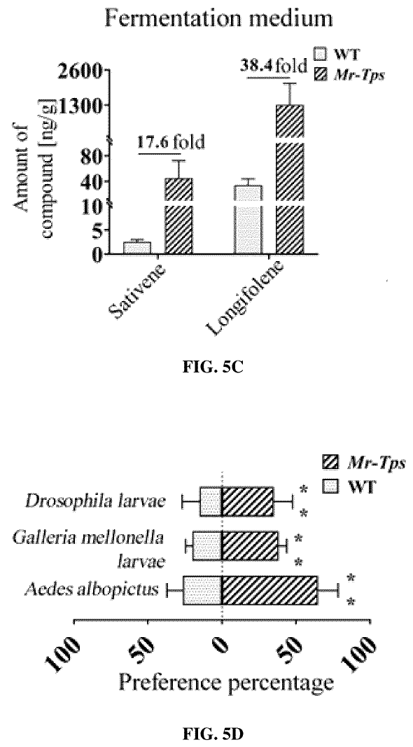

Further analysis found that compared with the muscardine cadavers derived from WT strain, the muscardine cadavers derived from Mr-Tps strain had a 193-fold and 28-fold increase in the volatilized amounts of longifolene and sativene, respectively, a 1.09-fold increase in geosmin, and a 1.48-fold increase in farnesene (FIG. 5A).

On the PDA medium, strain Mr-Tps produced 6 volatile compounds, of which a compound with the highest proportion was longifolene (Table 2). Compared with the WT strain, abilities of the strain Mr-Tps in volatilizing longifolene and sativene were increased by 98-fold and 4-fold, respectively, but the farnesene and geosmin were not detected (FIG. 5B).

Currently, Metarhizium spores are produced by industrialized mass production based on rice- and wheat bran-based fermentation media. On a similar fermentation medium, the transgenic strain Mr-Tps produced five volatile substances, of which longifolene had the highest proportion, followed by 1,3-octadiene (Table 2). Compared with the WT strain, abilities of the strain Mr-Tps in volatilizing longifolene and sativene were increased by 38.4-fold and 17.6-fold, respectively, but the farnesene and geosmin were not detected (FIG. 5C).

### Example 5 Expression of Tps Gene Improving Attraction of Metarhizium to Insects

According to the above device, the attraction of muscardine cadavers derived from the transgenic strain Mr-Tps and WT strain infections to the adult mosquitoes, Drosophila larvae and Galleria mellonella larvae, were compared; where the muscardine cadavers and freeze-to-death insects (control) of the device were replaced by the muscardine cadavers derived from the transgenic strain and WT strain infections, respectively. The preference percentage=[the number of insects selecting muscardine cadavers derived from transgenic strain (or WT strain) infections/the total number of insects] represented the attraction of the two strains to insects.

The results were shown in FIG. 5D. The Drosophila larvae, Galleria mellonella larvae, and adult mosquitoes significantly preferred the muscardine cadavers derived from transgenic strain Mr-Tps, compared with muscardine cadavers derived from WT.

According to the above device, the attraction of transgenic strain Mr-Tps and WT strain grown on PDA and on fermentation medium to mosquitoes was compared. The mycelia of transgenic strain Mr-Tps and WT strain grown on PDA and on fermentation medium for 14 d and with a fresh weight of 0.3 g were placed on both sides of the device, respectively, and a preference percentage was calculated after a period of treatment, where the preference percentage=the number of insects selecting mycelia of transgenic strain and WT strain/the total number of insects.

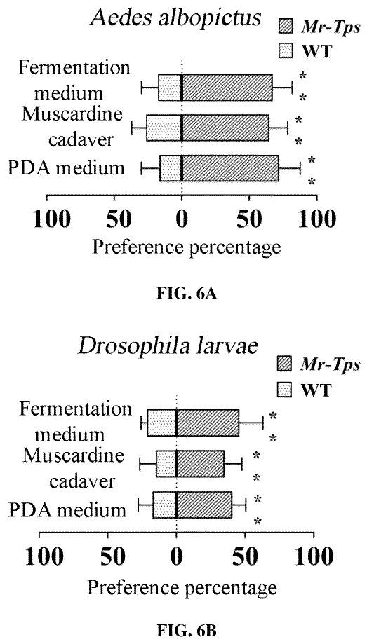

As shown in FIG. 6A and FIG. 6B, mosquito adults and Drosophila larvae significantly preferred the mycelium of the transgenic strain Mr-Tps compared to the mycelium of the WT.

### Example 6 Process for Controlling Mosquitoes with Transgenic Strains Based on Black Cloth Method

### 1) Methods

As described in the Background, when controlling mosquito adults, fungal spores were mainly placed on solid surfaces such as black cloth. To this end, it was tested whether the transgenic strain could also have stronger attraction on the black cloth. Metarhizium robertsii was cultured on PDA for 14 d, and then prepared into a spore suspension oil containing 8% vegetable oil and a total amount of 1×10⁸ spores by Triton-X-100 solution (0.01%) and the vegetable oil. The spore suspension oil was evenly sprayed on a surface of black gauze, air-dried, then placed in a cage of 1 m×1 m×1 m for alcohol disinfection, then added with 15 Aedes albopictus female adults for 5 d to 8 d without blood meal, where the experiment was conducted in the dark. After 12 h, the mosquitoes were collected one by one and placed into a 1.5 ml centrifuge tube containing 200 μl of 0.01% Triton-X-100; the mosquitoes were crushed with a sterilized grinding rod and spread evenly onto a Metarhizium screening medium (PDA containing 100 μg/mL ampicillin, 100 μg/mL kanamycin, 80 μg/mL streptomycin; 4 μg/mL dodine; and 10 μg/mL benomyl). The plate was incubated inverted at 26° C. for 5 d in dark; then the colony forming units (CFUs) on each plate were counted, to calculate an inoculation rate and an inoculation amount.

### 2) Results, Inoculation Rate, and Mortality

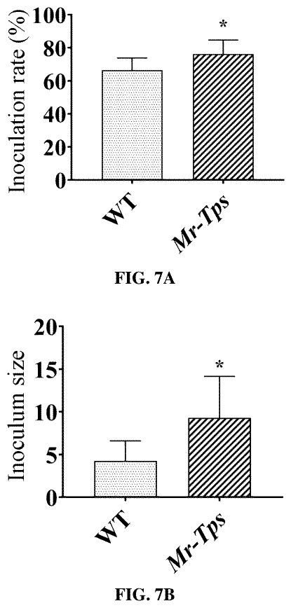

Referring to FIG. 7A and FIG. 7B, the results showed that in a cage of 1 m³, the inoculation rate to mosquitoes and the inoculation amount of per mosquito of the transgenic strain Mr-Tps each were significantly higher than those of the WT strain.

Although the above example has described the present disclosure in detail, it is only a part of, not all of, the examples of the present disclosure. Other examples may also be obtained by persons based on the example herein without creative efforts, and all of these examples shall fall within the claimed scope of the present disclosure.

1. A method for attracting insect and/or controlling pest comprising using sativene, the sativene has a structure shown in Formula I;

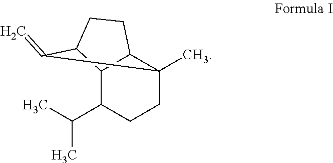

2. The method according to claim 1, wherein the insect comprises one or more of Drosophila, Galleria mellonella, and mosquitoes.

3. A recombinant biocontrol fungus, comprising a recombinant fungal expression plasmid, wherein the recombinant fungal expression plasmid is inserted with a pine longifolene synthesis gene.

4. The recombinant biocontrol fungus according to claim 3, wherein the pine longifolene synthesis gene has a nucleotide sequence set forth in SEQ ID NO: 1.

5. The recombinant biocontrol fungus according to claim 3, wherein an original fungus of the recombinant biocontrol fungus comprises Metarhizium.

6. A muscardine cadaver, wherein the muscardine cadaver was infected with the recombinant biocontrol fungus according to claim 3.

7. A method for attracting insect and/or controlling pest, the method comprising using the recombinant biocontrol fungus according to claim 3.

8. The method according to claim 7, wherein the insect comprises one or more of Drosophila, Galleria mellonella, and mosquitoes.

9. A method for preparing sativene, the method comprising using the recombinant biocontrol fungus according to claim 3, wherein the sativene has a structure shown in formula I;

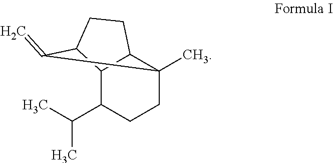

10. An insect attractant, wherein an active ingredient of the insect attractant comprises sativene or a recombinant biocontrol fungus or the muscardine cadaver according to claim 6; and the sativene has a structure shown in formula I;

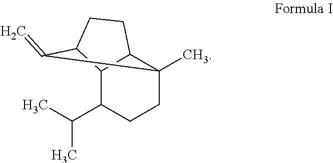

11. The muscardine cadaver according to claim 6, wherein the pine longifolene synthesis gene has a nucleotide sequence set forth in SEQ ID NO: 1.

12. The muscardine cadaver according to claim 6, wherein an original fungus of the recombinant biocontrol fungus comprises Metarhizium.

13. A method for attracting insect and/or controlling pest, comprising using the muscardine cadaver according to claim 6.
